(19) United States
(12) Patent Application Publication
(10) Pub. No.: US 2011/0040384 A1
(43) Pub. Date: Feb. 17, 2011

(54) IMPLANTABLE PROSTHETIC CAGE

(76) Inventors: Fredrick S. Junn, West Bloomfield, MI (US); Alex Kim, West Bloomfield, MI (US)

Correspondence Address: SCHOX PLC, 500 3rd Street, Suite 515, San Francisco, CA, 94107, US

(21) Appl. No.: 12/857,451

(22) Filed: Aug. 16, 2010

Related U.S. Application Data
(60) Provisional application No. 61/234,245, filed on Aug. 14, 2009.

(51) Int. Cl. A61F 2/44

(52) U.S. Cl. 623/17.12

(57) ABSTRACT

In one embodiment, the improved implantable prosthetic cage includes a hollow body, insertable into a human or any other animal, which includes an open receptacle adapted to hold a bone-growth promoting substance and at least one cover coupled to the body with a coupling mechanism to selectively seal the opening in the receptacle. The implantable prosthetic cage is preferably used for spinal fusion, but may be implanted in other parts of a human or other animal. In another embodiment, the method of utilizing an implantable prosthetic cage includes the steps of substantially sealing a bone-growth promoting substance within an implantable body, implanting the body between two bones while maintaining the seal and preventing contact between the substance and a bone, and disabling the seal, thereby allowing contact between the substance and a bone.

### CROSS-REFERENCE TO RELATED APPLICATIONS

This application claims the benefit of US Provisional Application No. 61/234,245, filed on 14 Aug. 2009, which is incorporated in its entirety by this reference.

### TECHNICAL FIELD

This invention relates generally to the surgical field, and more specifically to an improved implantable prosthetic cage in the medical implant field.

### BACKGROUND

Back pain is one of the most common afflictions among people, and contributes to a significant portion of physician visits. Treatments for back pain include medication, therapy, exercises, and one aggressive treatment method called spinal fusion. Spinal fusion is a surgical procedure in which two or more vertebrae (bones of the spine) are combined and fused together. Spinal fusion is typically used to treat patients with spinal abnormalities and/or severe chronic pain, such as patients with degenerative disc disease, spinal disc herniation, vertebral fracture, and scoliosis (curvature of the spine). Conventional methods of performing spinal fusion involve bone grafting, in which a bone tissue graft is typically placed inside a hollow intervertebral “cage” that is inserted between vertebrae to be fused, and allows the bone graft to act as a bridge to promote bone growth that fuses the vertebrae. The intervertebral cage includes holes that allow bone tissue to form around and through the cage to connect the vertebrae.

However, a relatively recent development in spinal fusion is the use of bone morphogenetic proteins (BMPs), a group of proteins that induce the formation of bone and cartilage. BMPs can be manipulated into a carrier such as a sponge, putty, fluid, powder, or other matrix that can be used to enhance or replace bone grafts in intervertebral cages in spinal fusion procedures. Use of BMPs in spinal fusion increases the speed and reliability of bone fusion, and induces quicker patient recovery. However, even with careful surgical technique, the BMP carrier may become displaced during insertion of the intervertebral cage. BMP displacement through the holes of the intervertebral cage is difficult to remove and can cause significant complications, such as unintended extension of the spinal fusion, spinal deformities, and pain or paralysis-inducing compression of the spinal cord. Thus, there is a need in the medical implant field to create an improved implantable prosthetic cage. This invention provides such an improved prosthetic cage.

### BRIEF DESCRIPTION OF THE FIGURES

FIG. 1 is a schematic representation of the hollow body of a preferred embodiment of the implantable prosthetic cage.

FIG. 2A is a schematic representation of the hollow body, cover, and coupling mechanism of a preferred embodiment in the unsealed state.

FIG. 2B is a schematic representation of the hollow body, cover, and decoupling mechanism of a preferred embodiment in the sealed state.

FIG. 3 is a schematic representation of a preferred embodiment with a second variation of the decoupling mechanism.

FIG. 4 is a schematic representation of a second preferred embodiment in the sealed state.

FIG. 5 is a schematic representation of a preferred method of utilizing an implantable prosthetic cage.

FIGS. 6A, 6B, 6C, and 6D are schematic representations of the steps of introducing a bone-growth substance into the implantable body (FIG. 6A), substantially sealing the substance within the body (FIG. 6B), implanting the body between two bones while maintaining the seal and preventing contact between the substance and a bone (FIG. 6C), disabling the seal thereby allowing contact between the substance and a bone (FIG. 6D).

FIGS. 7A and 7B is a schematic representation of a second preferred method of sealing and unsealing, respectively, the substance within the hollow body.

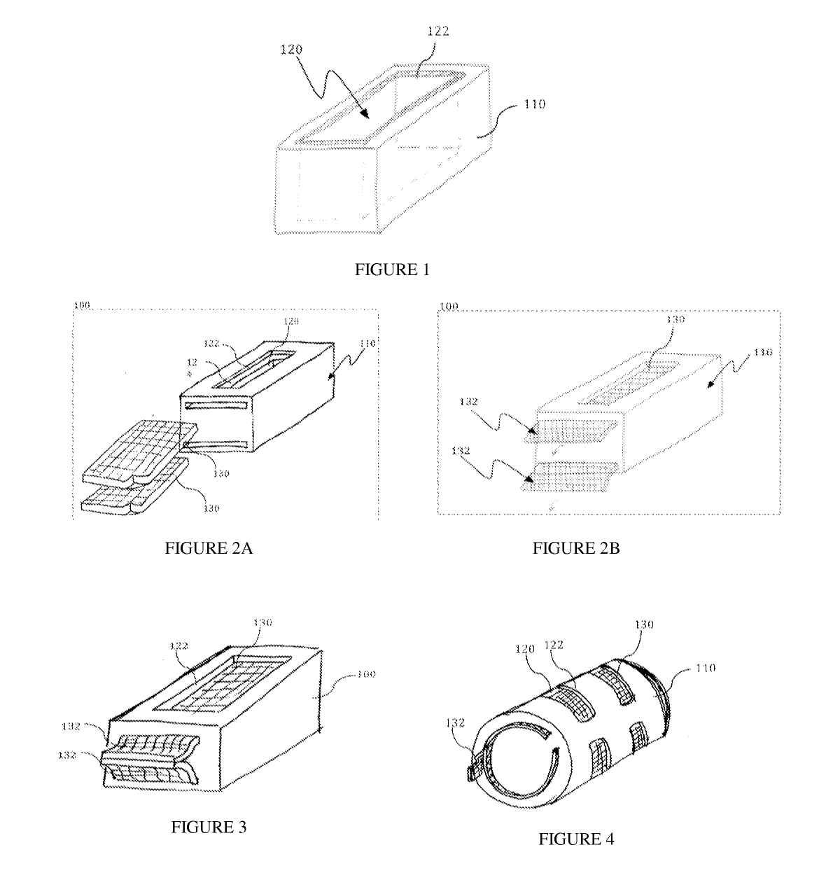

### DESCRIPTION OF THE PREFERRED EMBODIMENTS

The following description of the preferred embodiments of the invention is not intended to limit the invention to these preferred embodiments, but rather to enable any person skilled in the art to make and use this invention.

1. Implantable Prosthetic Cage

As shown in FIG. 2, the implantable prosthetic cage (or system) 100 comprises of a hollow body 110, insertable into a human or any other animal, which includes an open receptacle 120 adapted to hold a bone-growth promoting substance 140 and at least one cover 130 coupled to the body 110 with a coupling mechanism to selectively seal the opening in the receptacle 120. The implantable prosthetic cage 100 is preferably used for spinal fusion, but may be implanted in other parts of a human or other animal. Other applications include implantation between a fractured bone, such as the ulna or tibia, to help heal the fracture, or strategic implantation in a joint such as the ankle or wrist to fuse bones as a treatment for joint defects. The prosthetic cage may also be adapted to be an artificial hip replacement, in which the hollow body 110 is a long shaft that is implanted into a surgically-produced cavity in the proximal end of a femur of a patient, or adapted to be an artificial knee replacement, an artificial ankle replacement or a dental implant.

The hollow body 110 functions to hold a bone-growth promoting substance 140 and to selectively retain and direct the substance 140 in a controlled manner during implantation of the prosthetic cage. The shape of the hollow body 110 preferably approximates a rectangular prism and is adapted to be positioned on an anterior, posterior, and/or lateral side of the spinal cord between two vertebrae, but may alternatively be cylindrical, U-shaped, or any other suitable shape and size. The hollow body 110 is preferably rigid and made of PEEK or titanium, but may also be of varying stiffness and made of any suitable biocompatible material. The hollow body 110 may include features such as ridges, bumps, or spines that help retain the position of the prosthetic cage between two vertebrae after the prosthetic cage is implanted. Intervertebral cages are known and used by those skilled in the art, such as that described in U.S. Pat. No. 7,135,043 entitled “Intervertebral cage”, which is incorporated in its entirety by this reference.

The hollow body 110 preferably includes an open receptacle 120 that functions to contain and direct a substance 140 that promotes bone growth. The receptacle 120 is preferably a through hole with two openings 122, but may alternatively be a well with one opening, a porous enclosure with multiple openings 122, or any number of suitable receptacles 120 with any number of openings 122. The receptacle 120 is preferably configured to hold a viscous fluid, but may alternatively be adapted to hold solids such as bone chips or any suitable matter that accompanies implantation of the prosthetic cage.

The bone-growth promoting substance 140 that is contained in the receptacle 120 functions to promote directed bone growth. The substance 140 is preferably composed of a bone growth promoter and a carrier matrix. The bone growth promoter is preferably bone morphogenic protein (BMP), but may alternately be bone chips or activated stem cells. Use of BMP in intervertebral spacers is known to one skilled in the art, such as that described in U.S. Pat. No. 7,534,265 entitled “Intervertebral spacers with side wall accessible interior cavity”, which is incorporated in its entirety by this reference. The carrier matrix is preferably putty, but may alternately be a sponge or a fluid. The bone growth promoting substance 140 is preferably comprised of BMP in a putty matrix, such as OP-1 putty manufactured by Stryker. However, the bone growth promoting substance 140 may additionally be activated stem cells in a fluid or bone chips embedded in a polymer sponge.

The cover 130 functions to seal, close, obstruct, or otherwise block the receptacle opening 122 and enclose the bone-growth promoting substance 140 within the receptacle 120 during implantation. The cover 130 is preferably a sheet, but may alternatively be a pouch or a container the shape of the receptacle 120, and is preferably semi-rigid, but may alternatively be rigid or flexible. The cover 130 is preferably made of a biocompatible polymer such as PEEK, but may alternately be made of any biocompatible material such as titanium or silk, be made of a material that is dissolvable by solvent or be made of a bioresorbable material. The implantable prosthetic cage 100 preferably includes two covers 130, but may include one cover 130, multiple covers 130, or as many covers 130 as needed to cover the openings 122 of the body 110.

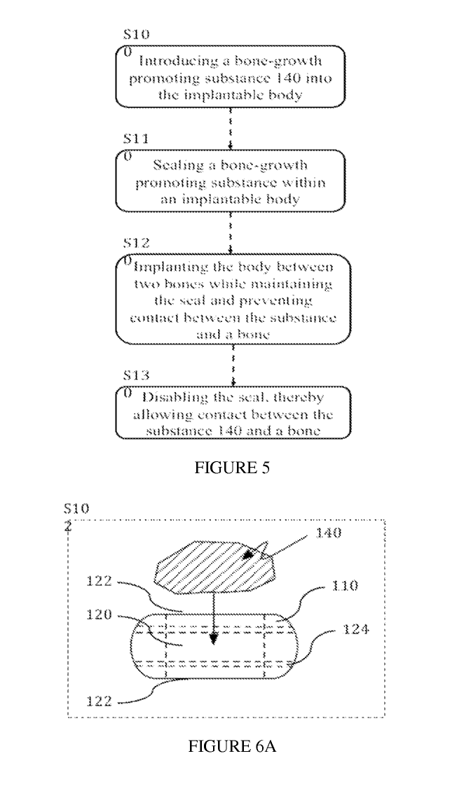

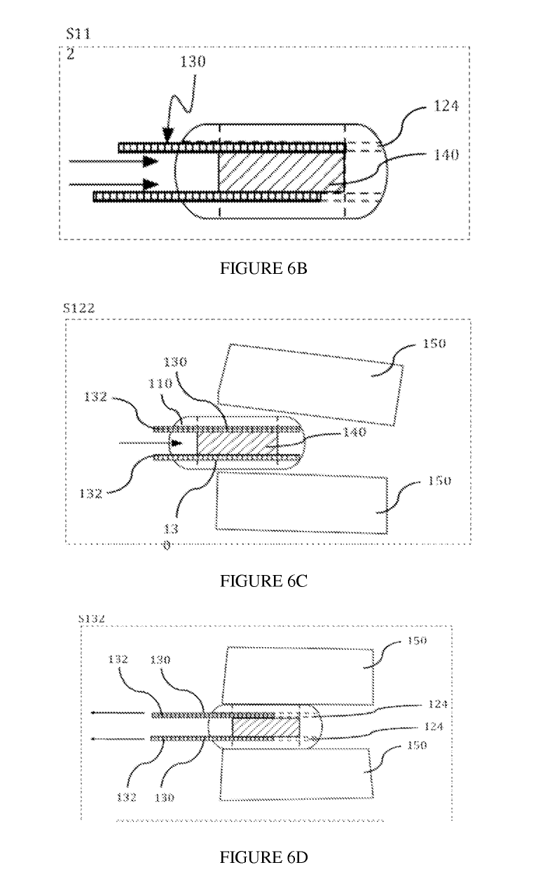

The coupling mechanism allows the cover 130 to selectively achieve a sealed (or “closed”) mode, wherein the cover 130 cooperates with the body 110 to form a seal that confines at least a portion of the substance 140 within the receptacle 120 of the body 110. The coupling mechanism preferably utilizes complimentary structures on the cover 130 and body 110 to mechanically connect the cover 130 with the body 110, but may also leverage material properties of the cover 130 and body 110, such as chemical or magnetic properties, to create a seal. The coupling mechanism preferably operates like a drawer, as shown in FIGS. 2 and 5, and includes a pair of parallel grooves 124 that border the opening on opposing walls of the receptacle 120. These grooves 124 extend the length of the opening such that sliding the edges of the cover 130 along the full length of the grooves 124 significantly covers 130 the opening. The cover 130 may alternatively wrap around the inside of the body 110 to cover the openings 122 as shown in FIG. 4, or may be coupled to the body 110 by an interference fit as shown in FIG. 6A, wherein the receptacle 120 is the same size or slightly smaller than the cover 130 such that the resultant contact friction maintains the cover 130 position. The cover 130 may also be coupled to the body 110 by adhering to the body 110, by screwing into the body 110, or by having micro-hooks that hook into micro-loops on the body 110. The cover 130 may alternatively be chemically active to react with a portion of the body 110, and forms a bond with the body 110 via covalent bonding, ionic bonding, or Van der Waals bonding. Alternatively, both the cover 130 and body 110 may have magnetic surfaces, and couple magnetically.

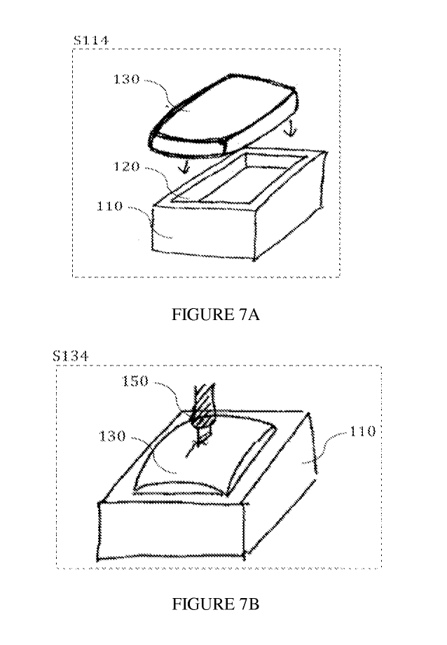

The decoupling mechanism allows the cover 130 to achieve an unsealed (or “open”) mode by creating an opening 122 in the seal that is formed between the cover 130 and body 110, thereby to allowing access to the substance 140 within the receptacle 120 after implantation of the prosthetic cage. The decoupling mechanism is preferably mechanical, but may be chemical as well, such as a solvent that solubilizes the cover 130, UV light that catalyzes a reaction to separate the cover 130 and the body 110, or naturally occurring enzymes that degrade the cover 130. The decoupling mechanism is preferably a tab 132 coupled to the cover 130, as shown in FIG. 2. Pulling on the tab displaces the cover 130 to uncover the receptacle opening 122. In embodiments with multiple covers 130, as shown in FIG. 3, the tabs 132 of the covers 130 may be connected to reduce the number of motions required to remove all the covers 130. The decoupling mechanism may alternatively be a scalpel 150, as shown in FIG. 7B, wherein puncturing the cover 130 with the scalpel 150 allows access to the substance 140 within the receptacle 120. The decoupling mechanism may also be a button coupled to the cover 130, wherein depression of the button results in the cover 130 retracting to uncover the opening. The decoupling mechanism is preferably activated by pulling, but may also be activated by pushing, perforating, applying a solvent, applying light or simply by implanting the prosthetic cage 100. Preferred tools to activate the decoupling mechanism are forceps, but may additionally be blades, screwdrivers, solvents, light, or magnets.

In a first preferred embodiment, as shown in FIG. 2, the implantable prosthetic cage 100 includes a rigid, rectangular body 110 that incorporates a through-hole receptacle 120 with two openings 122. The prosthetic cage further includes two plastic sheets as covers 130, one for each opening. The coupling mechanism includes two pairs of parallel grooves 124, located on opposing walls of the receptacle 120, with each pair bordering one receptacle opening 122 and extending the length of that respective opening. These grooves 124 are configured to receive and guide the edges of their respective covers 130 along the length of the opening, such that sliding the cover 130 through the full length of the grooves 124 will also slide the cover 130 the full length of the opening, thereby sealing the opening. The decoupling mechanism includes a tab 132 extending from each cover beyond the body 110. Pulling on the tabs 132 results in the covers 130 sliding along their respective grooves 124 to uncover their respective openings 122, thereby allowing access to the substance 140 contained within the receptacle 120.

In a variation of the first embodiment, as shown in FIG. 3, the tabs 132 of the first embodiment are joined together to reduce the number of motions required to decouple the cover 130 from the body 110. Pulling on the joined tabs 132 results in the covers 130 sliding along their respective grooves 124 to uncover their respective openings 122, allowing access to the substance 140 contained within the receptacle 120.

In a second preferred embodiment, as shown in FIG. 4, the implantable prosthetic cage 100 includes a rigid, cylindrical body 110 that incorporates a receptacle 120. The openings 122 of the receptacle 120 are on the curved surface of the body 110. The base 112 of the cylindrical body 110 includes a groove through the wall of the base 112 that traces the perimeter of the receptacle 120. In this embodiment, a single cover 130 is used. This cover 130 selectively seals the openings 122 by wrapping around the inside of the fluid receptacle 120, curving about the longitudinal axis. The cover 130 further includes a tab 132 extending beyond an edge of the body 110, such that pulling on the tab results in the cover 130 being withdrawn through the groove, thereby exposing the openings 122 of the receptacle 120. The wrapped cover 130 preferably has the cross-section of an arc or a circle, but may alternately have the cross section of a rectangle or any suitable cross-section that enables substantial sealing of the receptacle 120 openings 122.

In a third preferred embodiment, as shown in FIG. 6, the implantable prosthetic cage 100 includes a rigid, rectangular body 110 that incorporates a receptacle 120 in the shape of a well. The implantable prosthetic cage 100 further includes a cover 130 that is a sealed pouch made of biodegradable polymer, wherein the pouch contains the bone growth promoting substance 140. The coupling mechanism is an interference fit between the cover 130 and the body 110, and the cover 130 is slightly bigger than the receptacle 120. When the cover 130 is inserted into the receptacle 120, the friction between the cover 130 and receptacle 120 maintain the position of cover 130, and subsequently, the substance 140, within the receptacle 120. The decoupling mechanism is a scalpel 150 (or any other suitable device), and decoupling is accomplished by perforating the cover 130.

2. Method of Utilizing an Implantable Prosthetic Cage

The method of utilizing an implantable prosthetic cage, as shown in FIG. 5, preferably includes the steps of: substantially sealing a bone-growth promoting substance within an implantable body S110, implanting the body between two bones while maintaining the seal and preventing contact between the substance and a bone S120, and disabling the seal, thereby allowing contact between the substance and a bone S130. This method is preferably used to implant an intervertebral cage between two vertebrae of the spine, but may alternately be used to implant an artificial hip replacement, an artificial knee replacement, a dental implant, or any suitable prosthesis. This method preferably uses the apparatus described above, but may alternatively use any other suitable apparatus.

Step S110, which includes substantially sealing a bone-growth promoting substance within an implantable body, functions to form a seal that prevents migration of the substance out of the body during implantation. This step preferably includes fastening any number of covers over any number of openings in the body, thereby sealing the body and containing any substance introduced into the body before this sealing step within the body. As shown in FIG. 5, Step S110 preferably includes fastening a cover over an opening in the body and, as further shown in FIG. 5B, more preferably includes sliding a cover over the opening such that the cover substantially covers the opening S112. This may be accomplished by sliding the edges of the cover along a pair of parallel grooves that border the opening on opposing walls of the receptacle and extend the length of the opening. Other preferred methods of sealing the body include fitting a cover into the opening S114 (shown in FIG. 6A), or adhering the cover over the opening with adhesive.

Step S120, which includes implanting the body between two bones while maintaining the seal and preventing contact between the substance and a bone, functions to implant the prosthetic cage without undesired migration of the substance out of the prosthetic cage. This step is preferably performed after Step S110. The first portion of this step, which includes inserting the prosthetic cage between two bones, is known and used by those skilled in the art, as described in U.S. Pat. No. 6,648,915 entitled “Intervertebral cage and method of use”, which is incorporated in its entirety by this reference. However, the step of implanting the prosthetic cage may alternatively include any suitable step that depends on the specific application of the prosthetic cage. The second portion of this step, which includes maintaining the seal and preventing contact between the substance and a bone, is preferably accomplished by leaving the covers intact over the openings of the body during implantation.

Step S130, which includes disabling the seal, thereby allowing contact between the substance and a bone, functions to control when the substance is allowed to contact the bone. Step S130 is preferably performed after the implantation of the body between two bones S120, but may be performed before Step S120 as well. Step 130 preferably includes uncovering any openings on the body by pulling covers off of said openings S132, but may alternately include applying an external solvent to dissolve any covers over any openings in the body, perforating any covers over any openings in the body S134 (shown in FIG. 6B), or any other method of breaking the seal between the cover and the body. Step S132, which includes pulling covers off the openings of the body, as shown in FIG. 5, may further include pulling on tabs connected to the covers of the openings, such that pulling the tabs slides the covers off the openings to allow contact between the substance and bone. In one preferred method, the covers slide on parallel internal grooves that are located on opposing walls of the opening, near the edges of the opening.

The method of using an implantable prosthetic cage may additionally include the step of introducing a bone-growth promoting substance into the implantable body S100. This step is preferably performed before the step of “substantially sealing a bone-growth promoting substance within an implantable body,” and functions to enable the implantable body to better direct bone growth. Introducing a bone-growth promoting substance is preferably accomplished by packing a BMP-containing putty into a receptacle of the body, as shown in Step S102 in FIG. 5A, but may also be accomplished by soaking the body in the substance or by gelling a matrix containing bone-growth promoting substance within the body.

In a first preferred method of using the implantable prosthetic cage, as shown in FIG. 5, BMP-containing putty is packed into a through-hole receptacle in the body S102 and the body is sealed by sliding two covers over the opening of the receptacle S112. The prosthetic cage is implanted between two vertebrae S122, as described in the '915 patent. The seal is then disabled by pulling on tabs connected to the covers S132, thereby removing the covers from the implanted prosthetic cage to allow contact between the BMP-containing putty and the vertebrae.

In a second preferred method of using the implantable prosthetic cage, as shown in FIG. 7, a biodegradable cover enclosing BMP-containing gel is packed into a well in the body of the prosthetic cage S114 (shown in FIG. 7A). The prosthetic cage is implanted between two vertebrae S122, as described in the '915 patent. The seal is then disabled by perforating the cover S134 (shown in FIG. 7B) to allow contact between the BMP-containing putty and the vertebrae.

As a person skilled in the art will recognize from the previous detailed description and from the figures and claims, modifications and changes can be made to the preferred embodiments of the invention without departing from the scope of this invention defined in the following claims.

I claim:

1. An implantable prosthetic system, comprising:
a body, insertable between two bones, including a receptacle with an opening, wherein the opening and receptacle cooperate to receive and contain a bone-growth promoting substance; and
a cover, wherein the cover is selectively operable in two modes:
a sealed mode wherein the cover forms a seal with the body to prevent contact between the substance and a bone by confining the substance at least partially within the receptacle; and
an unsealed mode wherein the cover allows contact between the substance and a bone.

2. The system of claim 1, wherein the body includes a groove, and the cover couples to the body by sliding within said groove.

3. The system of claim 2 including a second groove parallel to the first groove, wherein said grooves are parallel to the opening and extend along the length of the opening.

4. The system of claim 3, wherein the sealed mode is achieved when the cover slides along the full length of the grooves such that the cover significantly obstructs the opening.

5. The system of claim 4, wherein the unsealed mode is achieved when the cover slides along the grooves such that the cover no longer significantly obstructs the opening.

6. The system of claim 5, wherein the cover is coupled to a tab that extends beyond the body to allow sliding of the cover.

7. The system of claim 4, wherein the receptacle is a hole through the body with a second opening; wherein the system further includes a second cover; and wherein the grooves are located on opposing walls of the receptacle.

8. The system of claim 2, wherein the cover is a plastic sheet.

9. The system of claim 1, wherein the cover is a pouch; and wherein the unsealed mode is achieved by puncturing the pouch.

10. An implantable spinal prosthetic system, comprising:
a rigid body, insertable between two vertebrae, including:
a receptacle having a first opening and a second opening, configured to receive a bone-growth promoting substance;
a pair of first grooves on opposing walls of the receptacle, near the opening, that span the length of the first opening; and
a pair of second grooves on opposing walls of the receptacle, near the opening, that span the length of the second opening;
a first cover slidingly engaged within the first grooves, such that the first cover slides along said first grooves to reversibly seal the first opening;
the first cover further including a tab that extends beyond the body to allow sliding of the first cover along the first grooves;
a second cover slidingly engaged within the second grooves, such that the second cover slides along said second grooves to reversibly seal the second opening;
wherein the first and second covers are selectively operable in two modes:
a closed mode wherein the first and second covers slide within the first and second grooves to significantly cover the first and second openings, respectively, such that the first and second covers form a seal that confines the substance within the receptacle, thereby preventing contact between the substance and bone; and
an open mode wherein the first and second covers are withdrawn to unseal the openings, thereby allowing contact between the substance and bone.

11. The system of claim 10, wherein the second cover is connected to the first cover to allow simultaneous displacement of the first and second covers along the first and second grooves, respectively.

12. A method for using an implantable spinal prosthetic system, comprising the steps of:
substantially sealing a bone-growth promoting substance within an implantable body;
implanting the body between two bones while maintaining the seal and preventing contact between the substance and a bone; and
disabling the seal, thereby allowing contact between the substance and a bone.

13. The method of claim 12, wherein the step of disabling the seal is performed after implanting the body between two bones.

14. The method of claim 12, further comprising the step of introducing a bone-growth promoting substance into the implantable body.

15. The method of claim 12, wherein the step of sealing the implantable body includes sliding a cover over an opening in the implantable body, such that the cover substantially covers the opening.

16. The method of claim 13, wherein the step of disabling the seal includes puncturing the cover.

17. The method of claim 12, wherein the step of disabling the seal includes sliding a cover off the implantable body.

18. The method of claim 17, wherein the step of sealing the implantable body includes sliding a cover over an opening in the implantable body, such that the cover substantially covers the opening.

19. The method of claim 18, wherein the steps of sealing and disabling the seal include sliding the cover along internal parallel grooves, located on opposing walls of the receptacle, that span the length of the opening.

20. The method of claim 19, wherein the step of disabling the seal includes pulling a tab coupled to the cover to slide the cover off the implantable body.
